(19) United States
(12) Patent Application Publication
(10) Pub. No.: US 2024/0247289 A1
(43) Pub. Date: Jul. 25, 2024

(54) ANTIBIOTIC FREE FERMENTATIONS, SYSTEMS AND METHODS FOR ACHIEVING ANTIBIOTIC FREE FERMENTATIONS, AND FERMENTATION BY-PRODUCTS GENERATED BY ANTIBIOTIC FREE FERMENTATIONS

(71) Applicant: POET Research, Inc., Sioux Falls, SD (US)

(72) Inventors: Benjamin Gacke, Baltic, SD (US); Stephen M. Lewis, Sioux Falls, SD (US)

(21) Appl. No.: 18/411,710

(22) Filed: Jan. 12, 2024

Related U.S. Application Data
(60) Provisional application No. 63/479,707, filed on Jan. 12, 2023.

(51) Int. Cl. C12P 7/06

(52) U.S. Cl. CPC C12P 7/06

(57) ABSTRACT

Methods, compositions, and systems for antibiotic-free propagation and fermentation using hops and formic acid, particularly large-scale operations for production of a bioproduct are provided. Hops and formic acid are combined in amounts sufficient to minimize growth of contaminating bacteria.

### RELATED APPLICATIONS

This application claims the benefit of U.S. Provisional Application No. 63/479,707 titled “Antibiotic Free Fermentations, Systems and Methods for Achieving Antibiotic Free Fermentations, and Fermentation By-Products Generated By Antibiotic Free Fermentations” filed Jan. 12, 2023, incorporated herein by reference.

### TECHNICAL FIELD

Provided herein are methods, systems, and compositions for propagation and fermentation in a biorefinery, for example, in large scale operations for production of ethanol and dried distiller's grain.

### BACKGROUND OF THE INVENTION

Fermentation processes utilize cultured microorganisms to convert carbon sources to a target bioproduct such as various alcohols, pharmaceuticals, proteins, etc. Example carbon sources include starch obtained from grain-based feedstocks (e.g., corn, sorghum/milo, barley, wheat, etc.), sugar obtained from sugar crops (e.g., sugar cane, sugar beets, etc.) and carbohydrate containing streams from pulp, agriculture and food processing (e.g., spent liquors, residues, waste streams, etc.) among others. Enzymes, whether endogenous to the grain, added to the fermenter, or produced by the primary microorganism or an adjunctive microorganism, convert the carbon source into a simple sugar that may be utilized by the microorganism. For example, amylases can convert starch to glucose. The microorganism then converts the simple sugar to the target bioproduct. For example, yeast, acting simultaneously with the enzymes, convert glucose to ethanol and carbon dioxide in a typical corn-to-ethanol biorefinery.

In a conventional cook process fermentation, slurried feedstock is cooked to liquefy starch in the feedstock. In a raw starch hydrolysis (RHS) process fermentation, the feedstock is not cooked and enzymes capable of raw starch hydrolysis are utilized.

A fermentation mash, whether in a cooked process or a RHS process, may become contaminated by bacteria that produce undesirable end-products. One substantial source for contaminant bacteria in a biorefinery, e.g., an ethanol production facility, is the feedstock. As much as 10,000 to 1,000,000 bacterial cells per gram enter the facility with the incoming grain. A majority of active contaminant bacteria belong to the classes of bacteria that can adapt and grow well in the biorefinery production conditions, e.g., lactic acid bacteria (LAB). Eventually, these contaminant lactic bacteria can become established in the production facility if proper measures for control are not taken.

Contaminant bacteria typically have a faster growth rate than the cultured microorganism, e.g., yeast, under ideal conditions. These bacteria, when they are in high numbers in the substrate, can begin to grow rapidly even before the cultured microorganism, e.g., yeast, is inoculated (added) to the mash. The contaminant bacteria can easily grow to levels that would produce enough end-products, e.g., lactic and acetic acids, to slow down the efficiency of target bioproduct, e.g., ethanol, produced by the cultured microorganism, e.g., yeast, ultimately leading to a loss in yield. The contaminant bacteria also consume carbon sources and other nutrients that would otherwise be converted to the target bioproduct by the cultured microorganism.

Many different microorganisms, including yeast and bacteria, may be cultured to produce a target bioproduct. Saccharomyces cerevisiae (S. cerevisiae) is widely used in producing various bioproducts, e.g., ethanol, butanol, pharmaceuticals, precursor chemicals, etc. Various bacteria have also been used or considered for culture including those that naturally produce the target bioproduct and those engineered to do so. Some examples include Escherichia coli (E. coli), lactic acid bacteria, e.g., Lactobacillus casei (L. casei), and Zymomonas mobilis (Z. mobilis) among others.

A number of strategies, including the use of low pH (between pH of 4.2 to 4.5) in fermentation, minimal use of antibiotics, and good plant management practices are used to keep the levels of contaminant bacteria to a small quantity. However, natural “non-antibiotic” solutions that eliminate the effects caused by contaminant bacteria are desirable.

The present invention is directed toward overcoming one or more of the problems discussed above.

### SUMMARY OF THE INVENTION

Provided herein are methods, systems, and compositions for propagation and fermentation, for example, fermentation used in the production of bioethanol.

Provided herein are methods of reducing contamination during ethanol production by treating various aspects of the fermentation, e.g., the slurry tank, the fermenter, and/or the propagator with hop acids and formic acid. The steps of the fermentation are performed in the absence of antibiotics, i.e., are antibiotic-free fermentations. In general, an antibiotic is a chemical used for treating bacterial infections in a human or animal body, while an antimicrobial is any additive that kills or inhibits growth of microorganisms inside and/or outside of the body.

Provided herein are methods of reducing contamination during ethanol production in the raw starch hydrolysis process by utilizing hop acids and formic acid in combination to combat lactic acid bacterial infection and contamination. The methods comprise (a) combining a feedstock, an ethanologen, hop acids, formic acid, and water in a propagator, slurry tank, and/or fermenter; and (b) fermenting the feedstock according to conditions provided herein. The steps of the fermentation are performed in the absence of antibiotics, i.e., are antibiotic-free fermentations.

Provided herein are methods of ethanol fermentation. In some embodiments, the method comprises (a) inoculating a feedstock with S. cerevisiae, and (b) fermenting the feedstock to produce ethanol. The feedstock can be, in some aspects, contaminated grain mash. The contaminated grain mash is treated with hop acids and formic acid in the propagator or fermenter. The feedstock can be, in some aspects, cooked, gelled, or liquified starch. The hop acids can be added to the cooled feedstock after cooking, and the formic acid can be added at any point prior to, during, or after cooking. Fermentations carried out with hop acids and formic acid can exhibit decreased levels of lactic acid bacteria relative to a fermentation performed with formic acid or hop acids alone. In some aspects, ethanol yield is increased relative to a fermentation performed with formic acid or hop acids alone.

Provided herein are methods of ethanol fermentation comprising: fermenting a feedstock with an ethanologen in the presence of formic acid and hop acids to produce ethanol and fermentation solids, wherein the fermentation is antibiotic free. The ethanologen can be a yeast or a bacterium. The feedstock can be any bacterially contaminated feedstock. In some instances, the feedstock is bacterially contaminated grain mash, such as that used in a raw starch fermentation. In some instances, the feedstock is cooked, then gelled, then liquified, for example, in preparation for fermentation.

In some aspects, the hop acids are present in a slurry tank, propagation tank, or fermentation tank at a range of about 10 ppm to about 150 ppm, +/−20%, about 10 ppm to about 130 ppm, +/−20%, about 20 ppm to about 30 ppm, +/−20%, or present in a concentration of about 12 ppm, 16 ppm, 17 ppm, 84 ppm, or 125 ppm, +/−20%.

In some aspects, the formic acid is present in a slurry tank, propagation tank, or fermentation tank at a range of about 10 ppm to about 500 ppm, +/−20%, or at 50 ppm, 60 ppm, 75 ppm, 100 ppm, 200 ppm, 300 ppm, or 400 ppm, +/−20%. In some aspects, the formic acid is added during fermentation fill at a concentration of about 200 ppm, +/−20%, and then diluted over time to 60 ppm, +/−20%.

The hop acids can be added before the formic acid, after the formic acid, or together with the formic acid.

In some embodiments, the fermentation is a raw starch hydrolysis process. In some aspects, the hop acids are added to a propagator with the ethanologen, to a slurry tank before the ethanologen is added, to a slurry tank after the ethanologen is added, and/or to a fermenter. In some aspects, the formic acid is added to a propagator with the ethanologen, to a slurry tank before the ethanologen is added, to a slurry tank after the ethanologen is added, and/or to a fermenter. In some aspects, the formic acid is added to the slurry tank after the hop acids. In some aspects, the ethanol yield is increased relative to a fermentation performed in the presence of formic acid alone or performed in the presence of hop acids alone. In some aspects, the level of lactic acid is decreased relative to a fermentation performed in the presence of formic acid alone or performed in the presence of hop acids alone.

In some embodiments, the fermentation is a cook process fermentation. In some aspects, the formic acid is added to the feedstock prior to cooking. In some aspects, the hop acids are added to the fermenter after the feedstock is cooled. In some aspects, the ethanol yield is increased relative to a fermentation performed in the presence of formic acid alone or performed in the presence of hop acids alone. In some aspects, the level of lactic acid is decreased relative to a fermentation performed in the presence of formic acid alone or performed in the presence of hop acids alone.

Provided herein are methods of antibiotic-free fermentation comprising (a) inoculating a feedstock with S. cerevisiae; (b) adding formic acid and hop acids, and (c) fermenting the feedstock.

Also provided herein are compositions comprising: bacterially contaminated grain mash, a yeast, formic acid, hop acids, and water. The compositions lack antibiotics.

Provided herein are compositions which are by-products of the described methods. In some aspects, the composition is antibiotic-free whole stillage, wet cake, mash, beer, thin stillage, syrup, dried distiller's grains with solubles (DDGS), and dried distiller's grains (DDG). In some aspects, the composition is an animal feed or a human food.

Further provided are systems for ethanol production. In some aspects, the systems comprise one or more fermenters comprising bacterially contaminated grain mash, an ethanologen, formic acid, hop acids, and water. In some aspects, the systems further comprise at least one of the following: a mill for preparation of feedstock; a propagator; and a distillation system. The systems are free of antibiotics.

### BRIEF DESCRIPTION OF THE DRAWINGS

FIG. 1 illustrates the antimicrobial effects of formic acid and hop acids in a RSH fermentation (raw starch fermentation) on % v/v ethanol at 88 hours. Antimicrobial ppm as depicted on the x-axis includes formic acid ppm, hop acids ppm (25 ppm in each instance where hop acids are included), and the combination of both formic acid and hop acids ppm.

FIG. 2 illustrates the antimicrobial effects of formic acid and hop acids in a RSH fermentation (raw starch fermentation) on ppm lactic acid at 88 hours. Antimicrobial ppm as depicted on the x-axis includes formic acid ppm, hop acids ppm (25 ppm in each instance where hop acids are included), and the combination of both formic acid and hop acids ppm.

FIG. 3 illustrates the antimicrobial effects of formic acid and hop acids in a cook process fermentation on % v/v ethanol at 72 hours. Antimicrobial ppm as depicted on the x-axis includes formic acid ppm, hop acids ppm (25 ppm in each instance where hop acids are included), and the combination of both formic acid and hop acids ppm.

FIG. 4 illustrates the antimicrobial effects of formic acid and hop acids in a cook process fermentation on ppm lactic acid at 72 hours. Antimicrobial ppm as depicted on the x-axis includes formic acid ppm, hop acids ppm (25 ppm in each instance where hop acids are included), and the combination of both formic acid and hop acids ppm.

FIG. 5 demonstrates how formic acid concentration in a slurry or propagation tank can be higher initially, and then diluted over several hours, in contrast to the steady concentration achieved in a laboratory.

FIG. 6 depicts contamination control performance achieved by antibiotic free fermentations. Typical fermentations last 60-80 hours. On the x-axis is batch number, the y-axis is the lactic acid concentration. The open circles indicate antibiotic free treatment, the closed circles are antibiotic treatment. Antibiotic free treatment includes hop acid and formic acid. Antibiotic treatment includes penicillin and virginiamycin. Black arrows indicate batches where the system was cleaned in place (CIP) with sodium hydroxide solution prior to batch fill. Points above black line indicate concerning levels of contamination.

FIG. 7, like FIG. 6, depicts contamination control using a different hop acid blend in the antibiotic free treatment.

### DESCRIPTION

As used in this specification and the appended claims, the singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

The word “exemplary” is used to mean serving as an example, instance, or illustration. Any embodiment or design described as “exemplary” is not necessarily to be construed as preferred or advantageous over other embodiments or designs, nor is it meant to preclude equivalent exemplary structures and techniques known to those of ordinary skill in the art. Rather, use of the word exemplary is intended to present concepts in a concrete fashion, and the disclosed subject matter is not limited by such examples.

The term “or” is intended to mean an inclusive “or” rather than an exclusive “or.” To the extent that the terms “comprises,” “has,” “contains,” and other similar words are used in either the detailed description or the claims, for the avoidance of doubt, such terms are intended to be inclusive in a manner similar to the term “comprising” as an open transition word without precluding any additional or other elements.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All patents, applications and non-patent publications mentioned in this specification are incorporated herein by reference in their entireties.

Industrial fermentation involves the breakdown of a feedstock by one or more microorganisms, e.g., yeast and/or bacteria, into one or more products. In addition to the feedstock, other nutrients may be provided to the organism to facilitate the fermentation. For example, a traditional ethanol fermentation process utilizes grain-based feedstocks (e.g., corn, sorghum/milo, barley, wheat, etc.), or other sugar sources (e.g., sugar cane, sugar beets, etc.). Enzymes, whether endogenous to the grain, added to the fermenter, or produced by yeast, convert components of the feedstock into simple sugars. The microorganism (also known as an ethanologen), acting subsequent to or simultaneously with the enzymes, convert the simple sugars to ethanol and carbon dioxide.

In a typical ethanol production plant, corn, or other suitable primary feedstock is ground for fermentation. This ground feedstock is referred to in some aspects as mash, for example, grain mash. The entire corn kernel can be ground for fermentation, or the corn kernel may be fractionated into its component parts, and only the starchy endosperm used in fermentation. Any suitable feedstock, subjected to virtually any suitable pretreatment, can be used in the methods and compositions provided herein.

The ground corn or other primary feedstock may be combined with water to form a slurry, and the pH of the slurry mixture may be adjusted as needed. A microorganism, for example, a yeast such as S. cerevisiae, is added. The amount of yeast starter employed is selected to effectively produce a commercially significant quantity of ethanol in a suitable time, e.g., less than 75 hours or less than 88 hours.

Yeast can be added to the fermentation by any of a variety of methods known for adding yeast to fermentation processes. Under aerobic conditions, yeast preferentially reproduces rather than producing ethanol. Under anaerobic conditions, yeast preferentially produces ethanol rather than reproducing. It is beneficial to propagate yeast under aerobic conditions to favor reproduction and build yeast cell volume and robustness. The propagated yeast may then be added to a fermenter. Other desired components can be added to the fermenter, including certain enzymes which produce monomeric sugars from polymeric sugars (e.g., glucose from starch) in the fermentable solids as in simultaneous saccharification and fermentation (SSF). These enzymes can be commercially sourced, may be present in the feedstock (genetically modified corn, for example), or may be expressed by the yeast or another microorganism co-cultured with the yeast. Exemplary enzymes include glucoamylase and alpha-amylase. Alternatively, saccharification can be performed separate from fermentation.

The slurry can be held at specified temperatures to facilitate the production of ethanol for a determined period of time. Fermenting can include contacting a mixture including sugars from the reduced plant material (e.g., fractionated plant material) with yeast under conditions suitable for growth of the yeast and production of ethanol. During fermentation, the yeast converts the sugars (e.g., glucose) to ethanol and carbon dioxide, and between the enzymatic production of sugars (e.g., glucose) and the fermentation process, sugars (e.g., glucose) may be maintained in the system at a low steady state. After fermentation, further treatment and/or distillation is performed to recover the ethanol, oil, carbon dioxide, dried distiller's grains (DDG), and/or other co-products.

In order to increase ethanol yield in a fermentation, native lactic acid bacteria that convert carbohydrates to organic acid must be reduced. Provided herein are methods to reduce bacterial contamination by adding formic acid and hop acids to the fermentation, i.e., an antibiotic free fermentation or propagation.

Hop acids are a class of compounds extracted from the hops plant. Alpha hop acids include humulone, adhumulone, cohumulone, posthumulone, and prehumulone. The most common iso-α-acids are cis- and trans-isohumulone. Hop acids benefit yeast by making the yeast more tolerant to the presence of ethanol, relieving replication stress, etc. It is contemplated herein that any alpha hop acids will be useful in the systems, methods, and compositions provided herein. Alpha hop acids are readily available. Exemplary hop acids include FermaHop Pro™, FermaHop X5™, and IsoStab™.

Formic acid is a simple carboxylic acid that cleaves proteins into peptides at the C- or N-terminal side of an aspartate residue. Formic acid is more potent than other weak acids, and has known anti-microbial activity. Formic acid is readily available as a commercial reagent.

Formic acid can be used as a feed additive due to its antimicrobial activity. However, formic acid is toxic to yeast and is known to inhibit ethanol production, and therefore its use in fermentation, prior to the present invention, was typically avoided. Amasil NA is an exemplary source of formic acid.

Hop acid products can be used for bacterial control in ethanol production, however at the required utilization rate the cost is high.

Provided herein are methods, systems, and compositions that utilize a combination of hop acids and formic acid in reducing lactic acid bacteria contamination. Surprisingly, the combination of hop acids and formic acid does not detrimentally impact ethanol production, and instead, can increase ethanol production relative to the use of either hop acids or formic acid alone.

The hop acids and formic acid can be added at various points prior to or during fermentation. It is not necessary to treat the entire fermentation volume. In some aspects, one or the other or both the formic acid and hop acids are added to a slurry tank. In some aspects, one or the other or both the formic acid and hop acids are added to a propagation tank. In some aspects, one or the other or both the formic acid and hop acids are added to a fermenter.

In some aspects, the hop acids are added to a propagator with the ethanologen, to a slurry tank before the ethanologen is added, to a slurry tank after the ethanologen is added, and/or to a fermenter.

In some aspects, the formic acid is added to a propagator with the ethanologen after addition of the hop acids, to a slurry tank before the ethanologen is added, to a slurry tank after the ethanologen is added, and/or to a fermenter.

The initial dose of the hop acids and formic acid can mitigate bacterial contamination on the feedstock. As the slurry tank is filled, the ethanologen grows more robustly and takes over as the predominant organism in the fermenter.

Illustratively, the hop acids can be added to a slurry tank or propagation tank and the contaminated grain mash is added and mixed with water. The ethanologen is added to the slurry tank, propagation tank, or the fermenter, and then the formic acid can be added to the slurry tank, propagation tank, or the fermenter. In such methods, the yeast is exposed to hop acids resulting in yeast health benefits and then sometime later (for example, 10 to 30 minutes later, or an hour or two later), formic acid is added.

In those instances where the fermentation is a cook process fermentation, the formic acid can be added to the feedstock prior to cooking or after cooking. The hop acids can be added to the fermenter after the feedstock is cooled.

Timing aspects in relation to the fermenter fill cycle can maximize treatment effects while minimizing costs. Nonetheless, the hop acids and formic acid can be added together or independently throughout the process, e.g., during fermentation, or before fermentation during early fill, during propagation, or while mixing the feedstock with the water and/or yeast. Slurry tanks are a known source of contamination, so treatment during mixing can mitigate contamination during fermentation.

In some aspects, the hop acids are dosed in a propagator. In some aspects, the hop acids are dosed in the slurry tank. In some aspects, the hop acids are added or present in the slurry tank or propagation tank at a range of about 50 ppm to about 150 ppm, +/−20%, for example, at a range of about 70 ppm to about 140 ppm, +/−20%, or about 80 ppm to about 130 ppm, +/−20%, or about 84 ppm, +/−20%, or about 125 ppm, +/−20%. In some aspects, the hop acids are dosed in the fermenter. In some aspects, the hop acids are added or present in the fermenter at a range of about 10 ppm (e.g., 12.5 ppm) to about 40 ppm, +/−20%, for example, at a range of about 20 ppm to about 30 ppm, +/−20%, or added/present at 25 ppm, +/−20%. The dose can vary, however, depending on the level of bacterial contamination in the slurry tank or on the feedstock.

In some aspects, the formic acid is dosed into the slurry or slurry tank. In some aspects, the formic acid is dosed into the propagation tank. In some aspects, the formic acid is dosed in the fermenter. In some aspects, the formic acid is added at a range of about 50 ppm to about 400 ppm, +/−20%, or added at 50 ppm, 75 ppm, 100 ppm, 200 ppm, 300 ppm, or 400 ppm, +/−20%. The dose can vary, however, depending on the level of bacterial contamination in the slurry tank or on the feedstock.

In some aspects, the rate of formic acid addition to a slurry tank, propagation tank, or fermenter is mitigated relative to the fill rate. A target concentration, for example, 200 ppm+/−20%, is added initially, and that same concentration is achieved in the slurry, propagation, and/or fermentation tanks. Eventually, the concentration can be decreased, for example, to 100 ppm, 90 ppm, 80 ppm, 70 ppm, 60 ppm, 50 ppm, 40 ppm, or 30 ppm, +/−20%. To maintain a given ppm, the formic acid can be added by pulsing or splitting dosages as the tanks fill. In some aspects, it is beneficial to have the formic acid concentration higher at the start of propagation. In some aspects, the formic acid is maintained at a level which is not inhibitory to yeast growth or ethanol production, e.g., is maintained at a concentration of 220 ppm or lower, or 210 ppm or lower, or 200 ppm.

Besides reducing LAB, the methods described herein are useful in reducing other wild-type detrimental bacteria including other Lactobacillus sp., as well as Lactococcus sp., Pediococcus sp., and Weissella sp.

Methods of fermentation can comprise the steps of any typical fermentation, whether in production of biofuels or foods and drinks for human and animal consumption. However, it is shown herein that adding hop acids and formic acid to a pre-fermentation vessel or fermenter allows for antibiotic-free food and drink products and livestock feeds.

Fermentations performed according to the methods provided herein can result in an ethanol yield that is increased relative to a fermentation performed in the presence of formic acid alone or performed in the presence of hop acids alone.

Fermentations performed according to the methods provided herein can result in decreased lactic acid relative to a fermentation performed in the presence of formic acid alone or performed in the presence of hop acids alone.

### Compositions

The methods described herein generate ethanol and by-products of ethanol fermentation. By performing antibiotic-free fermentations, the resulting by-products are antibiotic free.

Provided are compositions produced according to the methods disclosed herein, both for human consumption and animal consumption, which are antibiotic-free. The composition can be a human food product, e.g., any food containing grain products sourced from bacterially contaminated grains fermented in the absence of antibiotics. Exemplary food products include most processed foods containing grains, including flour, bread, cereals, noodles, infant foods, pancake mixes, beer, and malt. The composition can be an animal feed, for example, a feed for livestock such as beef cattle, dairy cattle, swine, sheep, goats, poultry, etc., a feed for horses, donkeys, ponies, mules, etc., a feed for ruminating zoo animals, or can be a feed for companion animals such as dogs, cats, birds, etc.

A livestock feed composition can comprise distiller's grain sourced from feedstock exposed to formic acid and hop acids in a slurry tank, propagator, and/or fermenter. A livestock feed composition can comprise antibiotic-free distiller's grains, such as malt distiller's grains, brewers' grains, condensed distiller's solubles, dried distillers solubles, distiller's wet grains, distiller's wet grains with solubles, gluten meal, and gluten feed.

Exemplary by-products include whole stillage, wet cake, mash, beer, thin stillage, syrup, dried distiller's grains with solubles (DDGS), and dried distiller's grains (DDG), etc. These antibiotic free by-products, i.e., antibiotic-free compositions, can be used as a livestock feed or supplement or as an ingredient in a livestock feed or supplement. In some aspects, the composition is fed to livestock. In some embodiments, the composition is used as a companion animal feed, supplement, or treat, or is used as an ingredient in a companion animal feed, supplement, or treat. In some aspects, the composition is fed to an animal. In some embodiments, the composition is used as a palatant. In some embodiments, the composition is used as an aquaculture feed or an ingredient in an aquaculture feed. In some aspects, the composition is fed to fish. In some embodiments, the composition is used as a human food or supplement or an ingredient in a human food or supplement. In other words, the composition is provided for human consumption. In some embodiments, the composition is used as a nutraceutical carrier or an ingredient in a nutraceutical carrier. In some embodiments, the composition is added to a growth media for culture of microorganisms. In some embodiments, the composition is used as a fertilizer or plant growth supplement, i.e., is added to soil or water to which the plant is exposed.

Other compositions include those found in a propagator, slurry tank, or fermenter. For instance, provided herein are compositions comprising a contaminated feedstock such as grain mash, water, an ethanologen, hop acids, and formic acid. These compositions can be present at various places prior to or during a fermentation. The hop acids and formic acid replace the antibiotic that would otherwise be needed to control bacterial contamination in the slurry tank or fermenter. Thus, the compositions are antibiotic free.

### Systems

Systems provided herein include systems typically used for ethanol production. Both cook process fermentations and raw starch fermentations benefit from the described treatment with hop acids and formic acid. Illustratively, the systems can comprise one or more fermenters comprising a bacterially contaminated grain mash, an ethanologen, formic acid, hop acids, and water. The systems can further comprise at least one of the following: a mill for preparation of feedstock; a propagator; and a distillation system. The systems are free of antibiotics.

### EXAMPLES

The following examples are provided for illustrative purposes only and are not intended to limit the scope of the invention.

### Example 1: Hop Acids Dosing

Various hops products can be used in the systems, methods, and compositions described herein. A hops dose response using FermaHop X5, IsoStab, or FermaHop Pro was performed in early fermentations to determine a useful amount of hop acids (provided as a range in ppm) according to the present disclosure. Each product has a different “active” concentration, thus, a range of 12-40 ppm each is a different total product concentration. The differences are summarized in Table 1.

| | Active | Active | Total |
|---|---|---|---|
| | Ingredient | Ingredient | Product |
| | Concentration | Range | Range |
| Product | (%) | Tested (ppm) | Tested (ppm) |
| FermaHop Pro ™ | 10 | 12-40 | 120-400 |
| FermaHop X5 ™ | 20 | 12-40 | 60-200 |
| IsoStab ™ | 30 | 12-40 | 40-133 |

FermaHop X5™, 25 ppm (which is equivalent to 125 ppm total product) was chosen for use in the experiments described below.

### Example 2: RSH Fermentation

### Preparation of Inoculum

Saccharomyces cerevisiae, yeast was added to a yeast propagation set up as follows. Ground corn was mixed with “pre-blend” (a mixture of the waters from various streams that are reused in an ethanol production facility) in an appropriate ratio to obtain slurry (mash) with 35 percent dry solids (including the solids contributed by the pre-blend water). “Preblend” was used as make up water in these studies to simulate the composition of mash, employing an exemplary embodiment provided herein, as it would be in a commercial ethanol production facility. The pH of the corn and “pre blend” slurry was approximately 4.5-4.8. An enzyme blend for raw starch hydrolysis was added to the slurry. Urea (50 percent liquor) was added to the slurry to give a final concentration of approximately 1000 ppm urea. The slurry was mixed in 100 milliliter wide mouth bottles at 60 milliliters per bottle. The bottles were covered with a bottle cap with a 1/16″ hole drilled into the top for gas release following addition of enzyme and the yeast and placed stationary in a water bath. The fermentation temperature was held constant at 32.2 degrees Celsius for 8 hours. This yeast propagation was then used to inoculate fermentations including yeast at a ratio level of 3-4% total fermentation volume.

### Preparation of Mash and Fermentation

Ground corn was mixed with “pre-blend” (a mixture of the waters from various streams that are reused in an ethanol production facility) in an appropriate ratio to obtain slurry (mash) with 34-38% percent dry solids (including the solids contributed by the pre-blend water). “Preblend” was used as make up water in these studies to simulate the composition of mash, as it would be in a commercial ethanol production facility. The pH of the corn and “pre blend” slurry was approximately 4.8. The enzyme blend for raw starch hydrolysis was added to the slurry. Urea (50 percent liquor) was added to the slurry to give a final concentration of approximately 50-100 ppm urea. The slurry was mixed in 100 milliliter wide mouth bottles at 60 milliliters per bottle. The bottles were covered with a bottle cap with a 1/16″ hole drilled into the top for gas release following addition of enzyme and inoculation of the necessary organisms and placed stationary in a water bath. The fermentation temperature was held constant at 30.6 degrees Celsius for 88 hours. Bottles were mixed well, and samples were withdrawn at various intervals during the course of fermentation and analyzed for sugars, organic acids and ethanol using high performance liquid chromatography (HPLC).

Hop acids were prepared at a 1:20 dilution and added at the target concentrations of 25 ppm. Phibro Xact (an antibiotic blend of penicillin and virginiamycin) was prepared at a 1:2000 dilution and added at the target concentration of 2.5 ppm. Formic Acid was prepared at a 1:20 dilution and added at the target concentrations of 100-400 ppm at 1 hour post yeast addition.

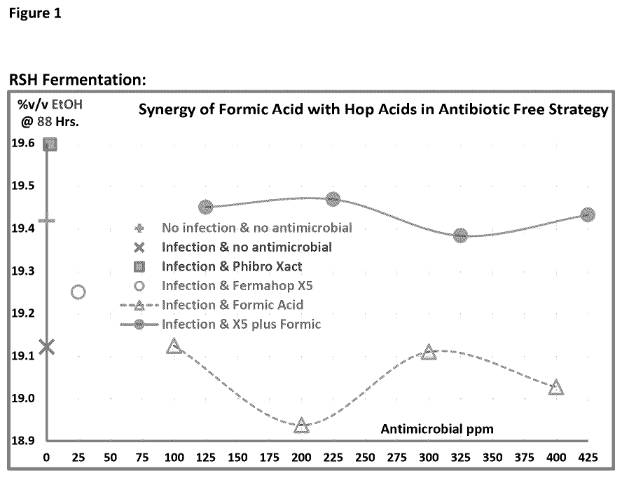

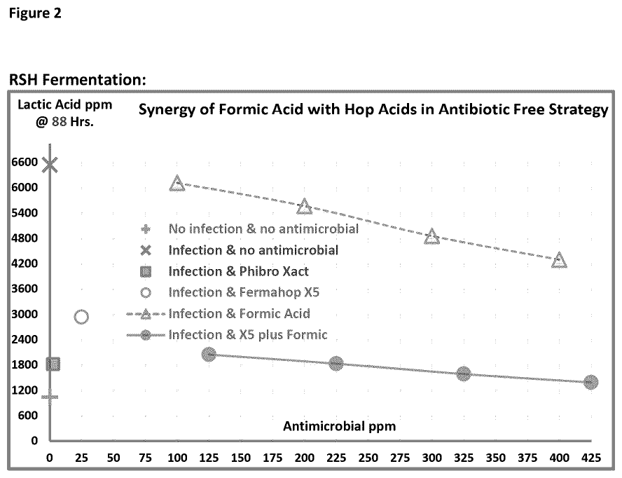

The results are provided in FIGS. 1 and 2. FIG. 1 shows that infected samples treated with formic acid (open triangles) substantially reduced % v/v ethanol compared to other infected samples, illustrating the toxicity of formic acid to yeast fermentations. Unexpectedly, infected samples treated with 100 ppm formic acid plus 25 ppm hops (125 ppm antimicrobial; first closed green circle) or 200 ppm formic acid plus 25 ppm hops (total 225 ppm antimicrobial; second closed green circle) produced more % v/v ethanol than uninfected samples without antimicrobial, and performed better than treatment with either formic acid or hops alone. FIG. 2 shows that including formic acid in the fermentation reduced lactic acid ppm at 88 hours fermentation in a dose dependent method, whether alone (open triangles) or in combination with hop acids (closed circles). Surprisingly, infected fermentation samples treated with formic acid doses 300 or 400 ppm in combination with hop acids (25 ppm) resulted in lower ppm lactic acid compared to infected fermentation samples treated with the commercial antibiotic, Phibro Xact™. In addition, the combination treatment outperformed treatment with either hop acids or formic acid alone.

### Example 3: Conventional (Cook Process) Fermentation

### Preparation of Inoculum

Saccharomyces cerevisiae, was added to a yeast propagation set up as follows. Liquefied corn slurry acquired from a biorefinery was diluted in an appropriate ratio to obtain slurry (mash) with 20 percent dry solids (including the solids contributed by the pre-blend water). The pH of the slurry liquefact was approximately 5.0-5.5. The enzyme blend for fermentation was added to the slurry. Urea (50 percent liquor) was added to the slurry to give a final concentration of approximately 1000 ppm urea. The slurry was dispensed in 100 milliliter wide mouth bottles at 60 milliliters per bottle. The bottles were covered with a bottle cap with a 1/16″ hole drilled into the top for gas release following addition of enzyme and inoculation of the necessary organisms and placed stationary in a water bath. The fermentation temperature was held constant at 32.2 degrees Celsius for 8-16 hours. This yeast propagation was then used to inoculate fermentations including yeast at a ratio level of 2% total fermentation volume.

### Preparation of Mash and Fermentation

Liquefied corn slurry was acquired from a biorefinery with 30-35% percent dry solids (including the solids contributed by the pre-blend water). The pH of the slurry was approximately 5.0. The enzyme blend for fermentation was added to the slurry. Urea (50 percent liquor) was added to the slurry to give a final concentration of approximately 1000 ppm urea. The slurry was mixed in 100 milliliter wide mouth bottles at 60 milliliters per bottle. The bottles were covered with a bottle cap with a 1/16″ hole drilled into the top for gas release following addition of enzyme and inoculation of the necessary organisms and placed stationary in a water bath. The fermentation temperature was held constant at 33 degrees Celsius for 72 hours. Bottles were mixed well, and samples were withdrawn at various intervals during the course of fermentation and analyzed for sugars, organic acids and ethanol using high performance liquid chromatography (HPLC).

Hop acids were prepared at a 1:20 dilution and added at the target concentrations of 25 ppm. Phibro Xact™ was prepared at a 1:2000 dilution and added at the target concentration of 2.5 ppm. Formic Acid was prepared at a 1:20 dilution and added at the target concentrations of 100-400 ppm at hour post yeast addition.

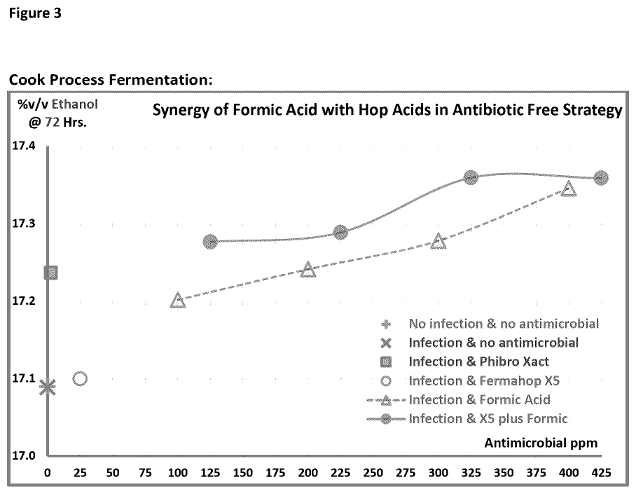

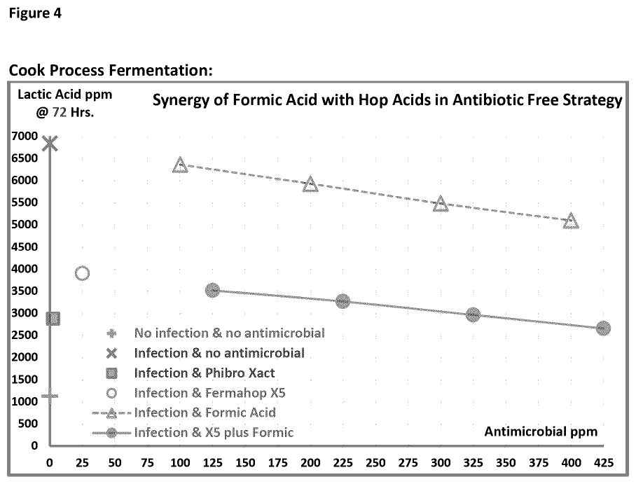

The results are provided in FIGS. 3 and 4. In samples from infected fermentations treated with formic acid, FIG. 3 shows that increasing doses of formic acid, alone (open triangles) or in combination with hop acids (closed circles), caused an increase in % v/v ethanol at 72 hours fermentation. Likewise, treatment with the combination of formic acid and hop acids performed better than treatment with either alone. Unexpectedly, at most dosages, the % v/v ethanol in the formic acid treated samples was greater than the % v/v ethanol generated by fermentation with Phibro Xact™. Ppm lactic acid decreased dose dependently in formic acid treated samples with hop acids (closed circles) and without hop acids (open triangles). However, the combination treatment decreased lactic acid to a greater extent than either hop acid or formic acid alone. See FIG. 4.

### Example 4: Scaled Propagation/Fermentation

### Process Overview

Corn flour and dilution waters were combined in a slurry tank to generate a pumpable liquid corn mash for fermentation. Typical additional inputs such as urea, starch degrading enzymes, acid for pH control, and antimicrobial agents were added into the slurry tank.

As the starting point of the process, the slurry tank is one of the key sites in the fermentation process for contamination control. Every 1-3 weeks the slurry vessel is emptied and cleaned in place (CIP) using a high temperature dilute sodium hydroxide (NaOH) solution. CIP frequency is determined by contamination rates in fermentation.

Yeast was used to ferment sugars from starch degradation into ethanol. As it is not economical to purchase and transport the amount of yeast needed in a typical fermenter, the yeast is grown on site in the yeast propagation vessel. Like the slurry tank, the yeast propagator is considered a critical control point for contamination. Yeast propagation batches are typically 6-14 hours. After each batch the vessel is cleaned in place.

The fermenter vessel is where the yeast from the yeast propagator and mash from slurry are combined. Typically, the fermenter is initially filled with a small volume of mash from slurry (1-5% tank volume) prior to transferring the yeast propagator. After the yeast propagator is transferred the tank resumes filling. A typical fermenter fill can take between 8-16 hours.

### Trial Overview

A trial to determine the feasibility of antibiotic free fermentation was completed at pilot scale in an 8-million-gallon ethanol per year facility.

Standard antimicrobial control involves the use of regular CIP and a combination of virginiamycin and penicillin antibiotic. Antibiotics at 0.75 ppm were dosed into slurry at the start of fermentation and 12 ppm dosed into the yeast propagator at the start of a batch. The antibiotics included PhibroXact™, a proprietary blend of antibiotics, or a combination of penicillin and virginiamycin.

For the antibiotic free batches, two ingredients were used to replace antibiotics for microbial control: hop acids and formic acid. Both hop acids (12.5 ppm) and formic acid (60 ppm) were added to the slurry tank at the start of fermentation. Formic acid was dosed using an automated dosing pump over the course of an hour to minimize any pH impacts of slug dosing the acid. Only hop acids were added to the yeast prop as formic acid can be inhibitory to yeast at high concentrations.

The antibiotic free trial was designed to run every-other batch, i.e., B1 antibiotics, B2 antibiotic free, B3 antibiotics, etc. This was done to mitigate the impact of any quality variations in the corn flour, dilution waters, or time between vessel CIP.

Trial outcomes were determined by organic acid production at the end of fermentation (80±5 hrs) as determined by HPLC. Bacteria counts at 20 hours into fermentation were also collected to get an indication of contamination levels at the start of the batch.

### Trial Results

For the first batch dosed with formic acid, bacteria counts were measured in the slurry tank before and after formic acid addition. Bacteria levels were reduced from 5.7×10\hat{ }5 CFU/ml to 2.3×10\hat{ }5 CFU ml, indicating that the formic acid was effective at microbial control.

During the trial, it became apparent that the timing of the hop acid and formic acid doses was important. Earlier dosing was desired such that the mash in the fermenter was clean prior to the yeast propagator transfer. Due to the extended fill hours of the batch, earlier dosing of the antimicrobial increased the effective concentration of the antimicrobial in the early hours of fermentation. Thus, the antimicrobial profile in the fermenter differed from the profile seen in the laboratory experiments that were used as a basis of dose selection in the trial. While frontloading the hop acid and formic acid dose is beneficial, a maximum level of formic acid was maintained in the fermenter to prevent yeast growth inhibition. Formic acid levels of 200 and 300 ppm at the time of yeast addition were tested in the trial, corresponding to a final concentration of 60 and 90 ppm at the end of fill.

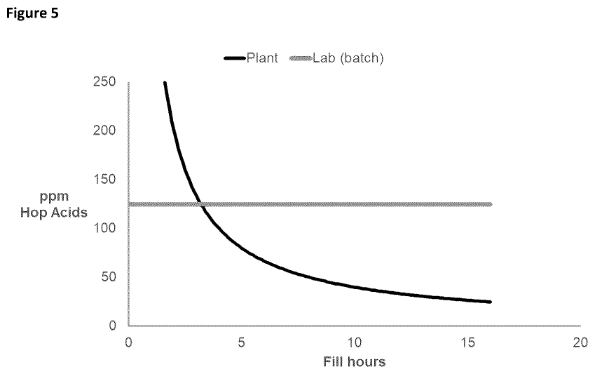

FIG. 5, which illustrates the front-loading of hop acid concentration in a slurry tank or propagation tank at an ethanol plant versus static concentration achievable in the lab. Over time, the hop acid concentration decreases.

Lactic acid concentrations are indicators of contamination in a fermenter. The level of lactic acid at the end of fermentation should be less than about 0.20% w/v. In the present study, antibiotic treated batches consistently demonstrated lower contamination (indicated by lower lactic acid) than the antibiotic free batches. And surprisingly, the there were no negative impacts observed on ethanol yield in the antibiotic free batches. Most antibiotic free batches were below 0.20% w/v validating the antibiotic free approach as a feasible operating strategy. It is contemplated that hop acid and formic acid doses along with CIP frequency can be adjusted within reasonable parameters and still maintain acceptable lactic acid levels. It is further contemplated that an acid tolerant yeast can be used as the ethanologen allowing lactic acid levels to rise above 0.20% w/v.

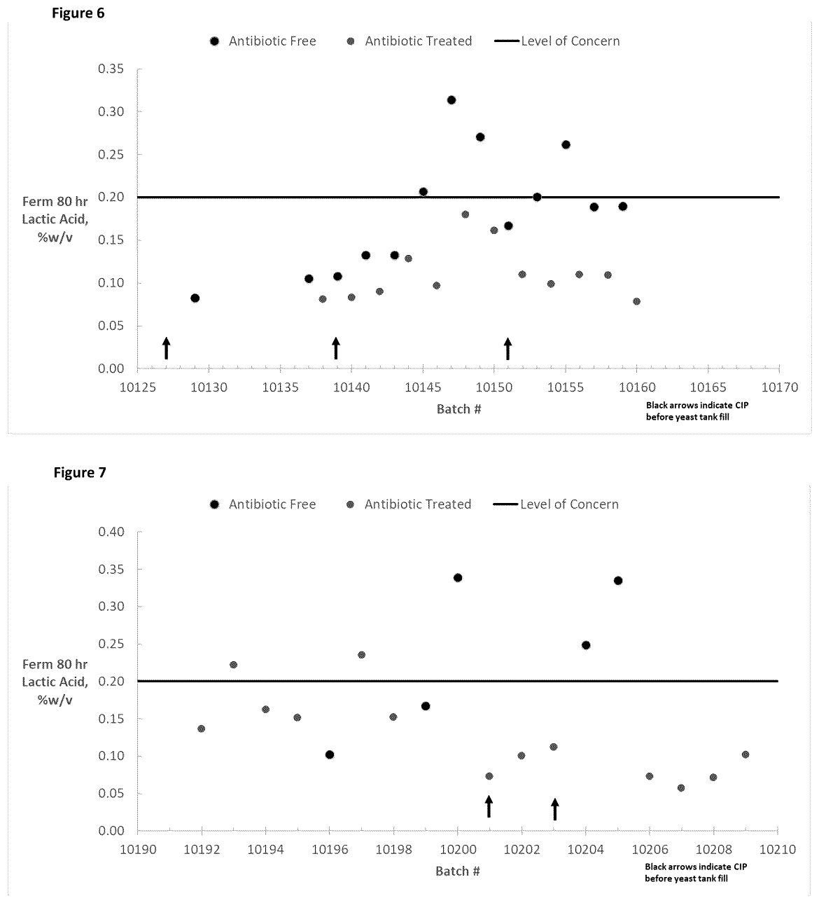

FIGS. 6 and 7 provide lactic acid levels after 80 hours fermentation for the antibiotic and antibiotic-free fermentation batches. As ethanol production remained unchanged in spite of the higher lactic acid levels in the antibiotic-free batches, this example confirms in a live fermentation that, surprisingly, the combination of hop acids and formic acid can mitigate bacteria to acceptable levels and not compromise ethanol production.

### Other Examples

1. A method of ethanol fermentation comprising: fermenting a feedstock with an ethanologen in the presence of formic acid and hop acids to produce ethanol and fermentation solids, wherein the fermentation is antibiotic free.
2. The method of example 1, wherein the ethanologen is a yeast or a bacterium.
3. The method of example 1, wherein the feedstock is bacterially contaminated grain mash.
4. The method of example 1, wherein the hop acids are added at a range of about 10 ppm to about 150 ppm, +/−20%, or present in a fermentation at about 12.5 ppm, +/−20%. or about 16 or 17 ppm, +/−20%, or present in a propagation at about 84 ppm, +/−20%, or 125 ppm, +/−20%.
5. The method of example 1, wherein the hop acids are added at a range of about 20 ppm to about 30 ppm, +/−20%, or added at 25 ppm, +/−20%.
6. The method of example 1, wherein the formic acid is added at a range of about 50 ppm to about 400 ppm, +/−20%, or added at 50 ppm, 75 ppm, 100 ppm, 200 ppm, 300 ppm, or 400 ppm, +/−20%.
7. The method of example 1, wherein the hop acids are added before the formic acid, after the formic acid, or together with the formic acid.
8. The method of example 1, wherein the fermentation is a raw starch hydrolysis process.
9. The method of example 8, wherein the hop acids are added to a propagator with the ethanologen, to a slurry tank before the ethanologen is added, to a slurry tank after the ethanologen is added, and/or to a fermenter.
10. The method of example 8, wherein the formic acid is added to a propagator with the ethanologen, to a slurry tank before the ethanologen is added, to a slurry tank after the ethanologen is added, and/or to a fermenter.
11. The method of example 8, wherein the formic acid is added to the slurry tank after the hop acids.
12. The method of example 1, wherein the fermentation is a conventional cook process fermentation.
13. The method of example 12, wherein the formic acid is added to the feedstock prior to cooking.
14. The method of example 12, wherein the hop acids are added to the fermenter after the feedstock is cooled.
15. The method of example 8, wherein the ethanol yield is increased relative to a fermentation performed in the presence of formic acid alone or performed in the presence of hop acids alone.
16. The method of example 8, wherein the level of lactic acid is decreased relative to a fermentation performed in the presence of formic acid alone or performed in the presence of hop acids alone.
17. The method of example 12, wherein the ethanol yield is increased relative to a fermentation performed in the presence of formic acid alone or performed in the presence of hop acids alone.
18. The method of example 12, wherein level of lactic acid is decreased relative to a fermentation performed in the presence of formic acid alone or performed in the presence of hop acids alone.
19. A method of antibiotic free fermentation comprising (a) inoculating a feedstock with S. cerevisiae; (b) adding formic acid and hop acids, and (c) fermenting the feedstock.
20. A composition comprising: bacterially contaminated grain mash, a yeast, formic acid, hop acids, and water.
21. A system for ethanol production comprising one or more fermenters comprising bacterially contaminated grain mash, an ethanologen, formic acid, hop acids, and water.
22. The system of example 21, further comprises at least one of the following: a mill for preparation of feedstock; a propagator; and a distillation system.
23. The system of example 21, which is antibiotic free.
24. A livestock feed sourced from a bacterially contaminated feedstock treated with formic acid and hop acids in a slurry tank, propagator, and/or fermenter.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

What is claimed is:

1. A method of ethanol fermentation comprising:
fermenting a feedstock with an ethanologen in the presence of formic acid and hop acids to produce ethanol and fermentation solids,
wherein the fermentation is antibiotic free.

2. The method of claim 1, wherein the ethanologen is a yeast or a bacterium.

3. The method of claim 1, wherein the feedstock is bacterially contaminated grain mash.

4. The method of claim 1, wherein the hop acids are present in a slurry tank, in a propagation tank, or a fermentation tank at a range of about 10 ppm to about 150 ppm, +/−20%.

5. The method of claim 1, wherein the hop acids are present in a slurry tank, in a propagation tank, or a fermentation tank at a range of about 20 ppm to about 30 ppm, +/−20%, or present at 16 ppm, 25 ppm, 84 ppm, or 125 ppm, +/−20%.

6. The method of claim 1, wherein the formic acid is present in a slurry tank, in a propagation tank, or a fermentation tank at a range of about 50 ppm to about 400 ppm, +/−20%, or present at 50 ppm, 60 ppm, 75 ppm, 100 ppm, 200 ppm, 300 ppm, or 400 ppm, +/−20%.

7. The method of claim 1, wherein the hop acids are added before the formic acid, after the formic acid, or together with the formic acid.

8. The method of claim 1, wherein the fermentation is a raw starch hydrolysis process.

9. The method of claim 8, wherein the hop acids are added to a propagator with the ethanologen, to a slurry tank before the ethanologen is added, to a slurry tank after the ethanologen is added, and/or to a fermenter.

10. The method of claim 8, wherein the formic acid is added to a propagator with the ethanologen, to a slurry tank before the ethanologen is added, to a slurry tank after the ethanologen is added, and/or to a fermenter.

11. The method of claim 8, wherein the formic acid is added to the slurry tank after the hop acids.

12. The method of claim 1, wherein the fermentation is a conventional cook process fermentation.

13. The method of claim 12, wherein the formic acid is added to the feedstock prior to cooking.

14. The method of claim 12, wherein the hop acids are added to the fermenter after the feedstock is cooled.

15. The method of claim 8, wherein the ethanol yield is increased relative to a fermentation performed in the presence of formic acid alone or performed in the presence of hop acids alone.

16. The method of claim 8, wherein levels of lactic acid are relative to a fermentation performed in the presence of formic acid alone or performed in the presence of hop acids alone.

17. The method of claim 12, wherein the ethanol yield is increased relative to a fermentation performed in the presence of formic acid alone or performed in the presence of hop acids alone.

18. The method of claim 12, wherein levels of lactic acid are relative to a fermentation performed in the presence of formic acid alone or performed in the presence of hop acids alone.

19. A method of antibiotic free fermentation comprising (a) inoculating a feedstock with S. cerevisiae; (b) adding formic acid and hop acids, and (c) fermenting the feedstock.

20. The method of claim 19, wherein the formic acid is added to a fermenter within 2 hours of yeast propagation.

21. The method of claim 20, wherein the formic acid is present in the fermenter at the end of fermenter fill to 60 ppm, +/−20%.

22. The method of claim 19, wherein the hop acids are added to a fermenter.

23. The method of claim 19, wherein the hop acids are present in the fermenter at a concentration of about 10 ppm to about 20 ppm, +/−20%.

24. The method of claim 19, wherein the hop acids are added to a slurry tank or propagation tank.

25. The method of claim 19, wherein the hop acids are present in a slurry tank or propagation tank at a concentration of about 50 ppm to about 130 ppm, +/−20%.

26. A composition comprising: bacterially contaminated grain mash, a yeast, formic acid, hop acids, and water.

27. A system for ethanol production comprising one or more fermenters comprising bacterially contaminated grain mash, an ethanologen, formic acid, hop acids, and water.

28. The system of claim 27, further comprises at least one of the following: a mill for preparation of feedstock; a propagator; and a distillation system.

29. The system of claim 27, which is antibiotic free.
